(19) United States
(12) Patent Application Publication
(10) Pub. No.: US 2025/0318806 A1
(43) Pub. Date: Oct. 16, 2025

(54) GRAPHICAL USER INTERFACE FOR VASCULAR STENT EXPANSION VISUALIZATION

(71) Applicant: Boston Scientific Scimed Inc., Maple Grove, MN (US)

(72) Inventors: Greta O'Brien, Minneapolis, MN (US); Mi Zhou, Maple Grove, MN (US); Judith Tiferes Wang, Shoreview, MN (US)

(73) Assignee: Boston Scientific Scimed Inc., Maple Grove, MN (US)

(21) Appl. No.: 19/172,989

(22) Filed: Apr. 8, 2025

Related U.S. Application Data
(60) Provisional application No. 63/632,904, filed on Apr. 11, 2024.

(51) Int. Cl. A61B 8/00; A61B 8/08; A61B 8/12; G06T 7/00; G06T 7/246; G06T 7/62; G06T 11/00

(52) U.S. Cl. CPC A61B 8/465; A61B 8/0841; A61B 8/12; A61B 8/467; G06T 7/0016; G06T 7/248; G06T 7/62; G06T 11/001; G06T 2200/24; G06T 2207/10132; G06T 2207/20081; G06T 2207/30104; G06T 2207/30196; G06T 2207/30204; G06T 2210/41

(57) ABSTRACT

The present disclosure provides a graphical user interface (GUI) arranged to convey information related to the IVUS images and stent expansion to a user. The GUIs can be generated to include a longitudinal depiction of the vessel and lumen borders as well as a deployed stent. A slider to navigate along the longitudinal axis of the vessel is provided where a stent expansion ratio is dynamically updated based on the slider location.

### CROSS-REFERENCE TO RELATED APPLICATIONS

This application claims the benefit of U.S. Provisional Application No. 63/632,904, filed Apr. 11, 2024, the disclosure of which is incorporated herein by reference in its entirety.

### TECHNICAL FIELD

The present disclosure generally relates to intravascular ultrasound (IVUS) imaging systems. Particularly, but not exclusively, the present disclosure relates to an improved graphical user interface for IVUS imaging systems in which expansion of a stent is visualized.

### BACKGROUND

Ultrasound devices insertable into patients have proven diagnostic capabilities for a variety of diseases and disorders. For example, intravascular ultrasound (IVUS) imaging systems have been used as an imaging modality for diagnosing blocked blood vessels and providing information to aid medical practitioners in selecting and placing stents and other devices to restore or increase blood flow.

IVUS imaging systems include a control module (with a pulse generator, image acquisition and processing components, and a monitor), a catheter, and a transducer disposed in the catheter. The transducer-containing catheter is positioned in a lumen or cavity within, or in proximity to, a region to be imaged, such as a blood vessel wall or patient tissue in proximity to a blood vessel wall. The pulse generator in the control module generates electrical pulses that are delivered to the transducer and transformed to acoustic pulses that are transmitted through patient tissue. The patient tissue (or other structure) reflects the acoustic pulses and reflected pulses are absorbed by the transducer and transformed to electric pulses. The transformed electric pulses are delivered to the image acquisition and processing components and converted into images displayable on the monitor.

IVUS systems can be used to image a vessel after a stent is deployed to confirm correct placement of the stent. Thus, there is a need for user interfaces, and particularly graphical user interfaces, that communicate information from the IVUS system to a user related to the deployment of the stent.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

In general, the present disclosure provides an improvement to computing devices and particularly to IVUS systems in that the present disclosure provides a graphical user interface arranged to convey the wealth of information with which modern IVUS systems generate. For example, an IVUS system may include machine learning features to process and analyze the signals generated during an IVUS run. Such information can include automatic detection of a lesion, key frames related to a lesion, a stent, or the like. With respect to detecting stents within a vessel, the present disclosure further provides to determine ratios of stent expansion at points along portions of the longitudinal axis of the stent and generate graphical information elements to display, as part of a graphical user interface (GUI), visualizations of the stent expansion ratios.

The improved IVUS systems and GUIs of the present disclosure can aid users of the IVUS systems in performing a percutaneous coronary intervention (PCI) procedure, and particularly, post-PCI procedures. For examples, the IVUS systems and GUIs provide information indicating how expanded the stent is in comparison to a healthy reference (e.g., healthy lumen, or the like), information indicating where areas of under-expansion are along the longitudinal axis of the stent, and/or information indicating whether the stent needs to be dilated post placement.

In some embodiments, the disclosure can be implemented as method for an intravascular ultrasound (IVUS) system. The method can comprise receiving, at a processor for an intravascular ultrasound (IVUS) system, a series of IVUS images of a vessel of a patient, the series of IVUS images comprising a plurality of frames; receiving, at the processor, an indication of a location of a stent in the vessel; receiving, at the processor, an indication of a lumen border; receiving, at the processor, designation of at least two of the plurality of frames as key frames; deriving, at the processor, an expansion ratio of the stent based at least on the key frames; generating, at the processor, a graphical user interface (GUI) comprising: a longitudinal vessel depiction comprising at least a portion of the lumen border and the stent, a distal bracket and a proximal bracket, a slider, a stent expansion ratio icon, and a visualization of the expansion ratio; and rendering the GUI for display on a display, wherein the longitudinal vessel depiction and the slider are disposed between the distal bracket and the proximal bracket, and wherein the stent expansion ratio icon is disposed at an intersection of the lumen border and the slider.

With further embodiments of the method, the slider comprises a location marker and a slider button, the location marker is a line substantially perpendicular to a longitudinal axis of the vessel, and the stent expansion ratio icon is disposed on the location marker at a point where the location marker intersects the lumen border.

With further embodiments of the method, the longitudinal vessel depiction comprises a longitudinal representation of a vessel border and the lumen border and a mirror of the longitudinal representation of the vessel border and the lumen border reflected about the longitudinal axis of the vessel.

With further embodiments of the method, the key frames comprise an end key frame and minimum area key frame and deriving the expansion ratio of the stent comprises deriving an area of the lumen at the end key frame; deriving an area of the stent at the minimum area key frame; and computing a quotient of the area of the stent divided by the area of the lumen.

With further embodiments of the method, the GUI comprises a visualization of a value of the expansion ratio.

With further embodiments of the method, the slider is disposed at a first point on the longitudinal axis of the vessel, the first point corresponds to the minimum area key frame, and the method further comprises receiving, at the processor from a user of the IVUS system, an indication to move the slider from the point to a second point on the longitudinal axis of the vessel, the second point different from the first point; deriving, at the processor, an updated expansion ratio of the stent based on an area of the stent at the second point and the area of the lumen; and generating, at the processor, an updated GUI, in the updated GUI the location marker of the slider intersects the lumen border at the second point on the longitudinal axis of the vessel, the stent expansion ratio icon is disposed at the intersection of the location marker and the lumen border, and the visualization of the value of the expansion ratio reflects the updated expansion ratio.

With further embodiments of the method, the end key frame is a distal key frame, the stent expansion ratio icon is a first stent expansion ratio icon, and the GUI comprises a second stent expansion ratio icon that is disposed at an intersection of the lumen border and the distal bracket.

With further embodiments of the method, the key frames comprise a proximal key frame and the method further comprises receiving, at the processor from a user of the IVUS system, an indication to utilize the proximal key frame in deriving the expansion ratio; deriving, at the processor, an updated expansion ratio of the stent based on the area of the stent at the minimum key frame and an area of the lumen at the proximal key frame; and generating, at the processor, an updated GUI, in the updated GUI the visualization of the value of the expansion ratio reflects the updated expansion ratio.

With further embodiments of the method, the visualization of the value of the expansion ratio depicts a percent.

With further embodiments of the method, the GUI depicts the lumen border in a first color, the vessel border in a second color different from the first color, and the stent expansion ratio icon in the second color.

With further embodiments of the method, receiving the indication of the location of the stent in the vessel comprises inferring for each frame of the plurality of frames, whether a stent is represented in the frame using a machine learning (ML model).

With further embodiments of the method, receiving the indication of the lumen border comprises identifying for each frame of the plurality of frames, a diameter of the lumen border at the frame.

With further embodiments of the method, receiving designations of at least two of the plurality of frames as key frames comprises identifying as a distal key frame a one of the plurality of frames distal to a most distal one of the plurality of frames in which the stent is represented; identifying as a proximal key frame a one of the plurality of frames proximal to a most proximal one of the plurality of frames in which the stent is represented; deriving a lumen area for each frame of the plurality of frames between the distal key frame and the proximal key frame; and identifying as a minimum area key frame a one of the plurality of frames between the distal key frame and the proximal key frame with the smallest lumen area.

In some embodiments, the disclosure can be implemented as an intra vascular ultrasound (IVUS) system. The IVUS system can comprise a processor coupled to a memory, the memory comprising instructions executable by the processor, the processor configured to couple to an intravascular ultrasound (IVUS) system and configured to execute the instructions, which instructions when executed cause the processor to implement any of the methods described herein.

In some embodiments, the disclosure can be implemented as at least one machine readable storage device. The at least one machine readable storage device can comprise a plurality of instructions that in response to being executed by a processor of an intravascular ultrasound (IVUS) system cause the processor to implement any of the methods described herein.

In some embodiments, the disclosure can be implemented as an intravascular ultrasound (IVUS) system. The IVUS system can comprise a processor; and a memory storage device coupled to the processor. The memory storage device can comprise instructions executable by the processor, which when executed cause the processor to receive a series of IVUS images of a vessel of a patient, the series of IVUS images comprising a plurality of frames, receive an indication of a location of a stent in the vessel, receive an indication of a lumen border, receive designation of at least two of the plurality of frames as key frames, derive an expansion ratio of the stent based at least on the key frames, generate a graphical user interface (GUI) comprising a longitudinal vessel depiction comprising at least a portion of the lumen border and the stent, a distal bracket and a proximal bracket, a slider, a stent expansion ratio icon, and a visualization of the expansion ratio, and render the GUI for display on a display, the longitudinal vessel depiction and the slider are disposed between the distal bracket and the proximal bracket, and the stent expansion ratio icon is disposed at an intersection of the lumen border and the slider.

With further embodiments of the IVUS system, the slider comprises a location marker and a slider button, the location marker is a line substantially perpendicular to a longitudinal axis of the vessel, and the stent expansion ratio icon is disposed on the location marker at a point where the location marker intersects the lumen border.

With further embodiments of the IVUS system, the longitudinal vessel depiction comprises a longitudinal representation of a vessel border and the lumen border and a mirror of the longitudinal representation of the vessel border and the lumen border reflected about the longitudinal axis of the vessel.

With further embodiments of the IVUS system, the key frames comprise an end key frame and minimum area key frame and the instructions when executed by the processor to derive the expansion ratio of the stent cause the processor to derive an area of the lumen at the end key frame; derive an area of the stent at the minimum area key frame; and compute a quotient of the area of the stent divided by the area of the lumen.

With further embodiments of the IVUS system, the GUI comprises a visualization of a value of the expansion ratio.

With further embodiments of the IVUS system, the slider is disposed at a first point on the longitudinal axis of the vessel, the first point corresponds to the minimum area key frame, and the instructions when executed by the processor further cause the processor to receive, from a user of the IVUS system, an indication to move the slider from the point to a second point on the longitudinal axis of the vessel, the second point different from the first point; derive an updated expansion ratio of the stent based on an area of the stent at the second point and the area of the lumen; and generate an updated GUI, in the updated GUI the location marker of the slider intersects the lumen border at the second point on the longitudinal axis of the vessel, the stent expansion ratio icon is disposed at the intersection of the location marker and the lumen border, and the visualization of the value of the expansion ratio reflects the updated expansion ratio.

With further embodiments of the IVUS system, the end key frame is a distal key frame, the stent expansion ratio icon is a first stent expansion ratio icon, and the GUI comprises a second stent expansion ratio icon that is disposed at an intersection of the lumen border and the distal bracket.

With further embodiments of the IVUS system, the key frames comprise a proximal key frame and the instructions when executed by the processor further cause the processor to receive, from a user of the IVUS system, an indication to utilize the proximal key frame in deriving the expansion ratio; derive an updated expansion ratio of the stent based on the area of the stent at the minimum key frame and an area of the lumen at the proximal key frame; and generate an updated GUI, wherein in the updated GUI the visualization of the value of the expansion ratio reflects the updated expansion ratio.

With further embodiments of the IVUS system, the visualization of the value of the expansion ratio depicts a percent.

In some embodiments, the disclosure can be implemented as at least one machine readable storage device. The at least one machine readable storage device can comprise a plurality of instructions that in response to being executed by a processor of an intravascular ultrasound (IVUS) system cause the processor to receive a series of IVUS images of a vessel of a patient, the series of IVUS images comprising a plurality of frames; receive an indication of a location of a stent in the vessel; receive an indication of a lumen border; receive designation of at least two of the plurality of frames as key frames; derive an expansion ratio of the stent based at least on the key frames; generate a graphical user interface (GUI) comprising: a longitudinal vessel depiction comprising at least a portion of the lumen border and the stent, a distal bracket and a proximal bracket, a slider, a stent expansion ratio icon, and a visualization of the expansion ratio; and render the GUI for display on a display, wherein the longitudinal vessel depiction and the slider are disposed between the distal bracket and the proximal bracket, and wherein the stent expansion ratio icon is disposed at an intersection of the lumen border and the slider.

With further embodiments of the at least one machine readable storage device, the slider comprises a location marker and a slider button, the location marker is a line substantially perpendicular to a longitudinal axis of the vessel, and the stent expansion ratio icon is disposed on the location marker at a point where the location marker intersects the lumen border.

With further embodiments of the at least one machine readable storage device, the longitudinal vessel depiction comprises a longitudinal representation of a vessel border and the lumen border and a mirror of the longitudinal representation of the vessel border and the lumen border reflected about the longitudinal axis of the vessel.

With further embodiments of the at least one machine readable storage device, the GUI depicts the lumen border in a first color, the vessel border in a second color different from the first color, and the stent expansion ratio icon in the second color.

With further embodiments of the at least one machine readable storage device, the instructions when executed by the processor to receive the indication of the location of the stent in the vessel cause the processor to infer for each frame of the plurality of frames, whether a stent is represented in the frame using a machine learning (ML model).

With further embodiments of the at least one machine readable storage device, the instructions when executed by the processor to receive the indication of the lumen border cause the processor to identify for each frame of the plurality of frames, a diameter of the lumen border at the frame.

With further embodiments of the at least one machine readable storage device, the instructions when executed by the processor to receive designations of at least two of the plurality of frames as key frames cause the processor to identify as a distal key frame a one of the plurality of frames distal to a most distal one of the plurality of frames in which the stent is represented; identify as a proximal key frame a one of the plurality of frames proximal to a most proximal one of the plurality of frames in which the stent is represented; derive a lumen area for each frame of the plurality of frames between the distal key frame and the proximal key frame; and identify as a minimum area key frame a one of the plurality of frames between the distal key frame and the proximal key frame with the smallest lumen area.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1 illustrates an IVUS system, in accordance with at least one embodiment of the present disclosure.

FIG. 2 illustrates another IVUS system, in accordance with at least one embodiment of the present disclosure.

FIG. 3 illustrates a graphical user interface (GUI) that can be generated by an IVUS system, in accordance with at least one embodiment of the present disclosure.

FIG. 4 illustrates another GUI that can be generated by an IVUS system, in accordance with at least one embodiment of the present disclosure.

FIG. 5 illustrates an enlarged view of a portion of the GUI of FIG. 4.

FIG. 6A, FIG. 6B, FIG. 6C, and FIG. 6D illustrate a portion of a GUI that can be generated by an IVUS system and dynamic updates to the portion of the GUI, in accordance with at least one embodiment of the present disclosure.

FIG. 7 illustrates a logic flow to generate and/or update a GUI for an IVUS system, in accordance with at least one embodiment of the present disclosure.

FIG. 8 illustrates a computer-readable storage medium, in accordance with at least one embodiment of the present disclosure.

FIG. 9 illustrates computing device, in accordance with at least one embodiment of the present disclosure.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and operation, together with further objects and advantages will be better understood from the following description when considered in connection with the accompanying figures. It is to be expressly understood, however, that each of the figures is provided for the purpose of illustration and description and is not intended as a definition of the limits of the present disclosure.

As noted, the present disclosure relates to IVUS systems and to determining ratios of stent expansion and providing visualizations of the ratios of stent expansion. In particular, the disclosure provides a graphical user interface (GUI) arranged to convey information related to a vessel and a stent placed in the vessel via a PCI procedure. To that end, an example IVUS imaging system and patient vessel are briefly described prior to discussing details of the present disclosure.

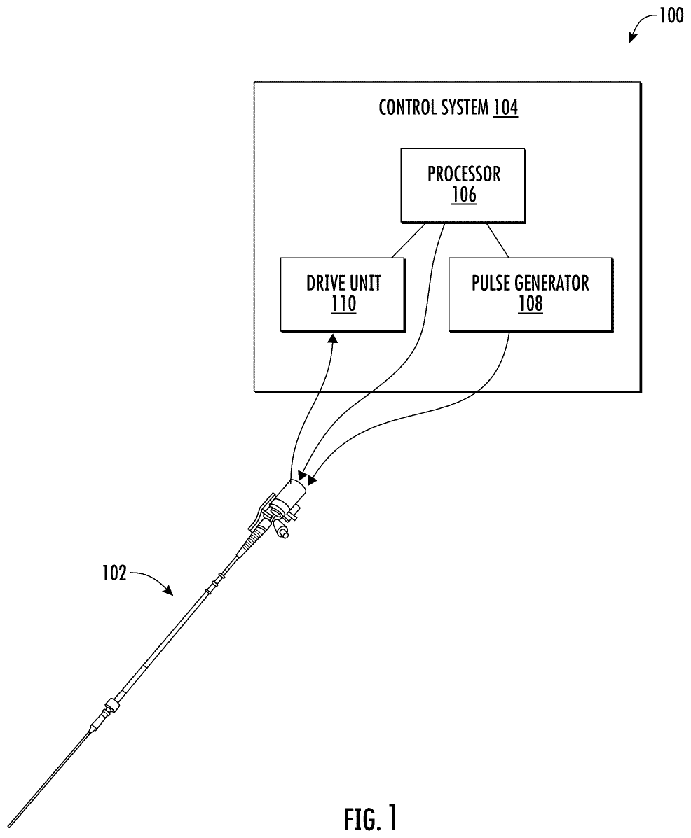

Suitable IVUS imaging systems include, but are not limited to, one or more transducers disposed on a distal end of a catheter configured and arranged for percutaneous insertion into a patient, and particularly into a vessel of a patient. FIG. 1 illustrates an example IVUS imaging system 100. The IVUS imaging system 100 includes a catheter 102 that is couplable to a control system 104. The control system 104 may include, for example, a processor 106, a pulse generator 108, and a drive unit 110.

The catheter 102 can comprise an imaging core having one or more transducers (not shown). The pulse generator 108 forms electric pulses that may be applied to the one or more transducers of the imaging core. Mechanical energy from the drive unit 110 can be used to drive the imaging core (e.g., via a drive cable disposed in the catheter 102) rotationally. The transducer can convert the electrical pulses into acoustic energy, a portion of which may be reflected from vessel tissue and/or other objects (e.g., a stent, or the like). The reflected acoustic signals can be received by the transducer and converted into electrical signals. These electrical signals converted by the transducer responsive to receiving the reflected acoustic energy can be communicated to the processor 106.

The processor 106 can process these electrical signals to form a series of images indicative of the interior of the vessel. For example, a scan converter can be used to map scan line samples (e.g., radial scan line samples, or the like) to a two-dimensional Cartesian grid, which can be used as the basis for a series of IVUS images that can be displayed for a user. The processor 106 may also be used to control the functioning of one or more of the other components of the control system 104. For example, the processor 106 may be used to control at least one of the frequency or duration of the electrical pulses transmitted from the pulse generator 108, the rotation rate of the imaging core by the drive unit 110. Additionally, where IVUS imaging system 100 is configured for automatic pullback, processor 106 can send control signals to the drive unit 110 to control the velocity and/or length of the pullback.

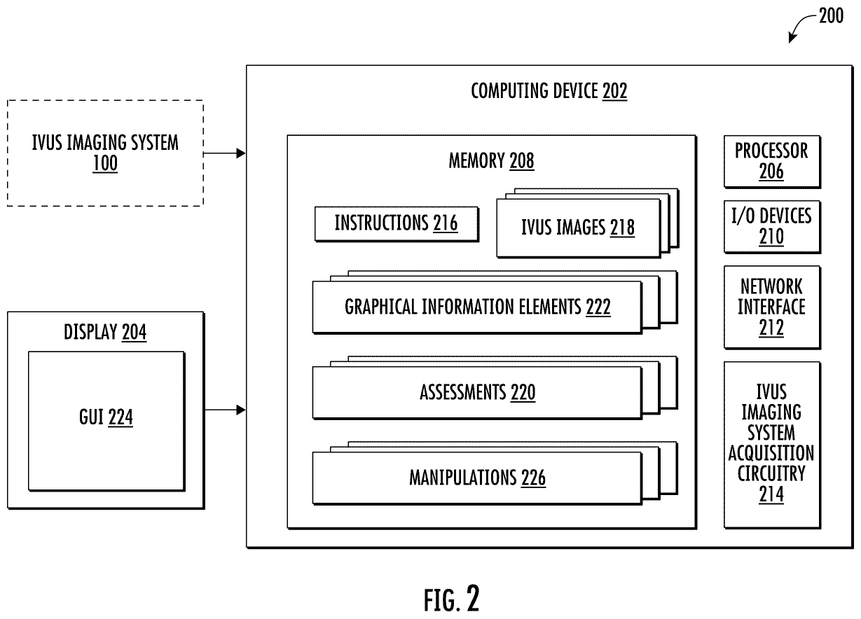

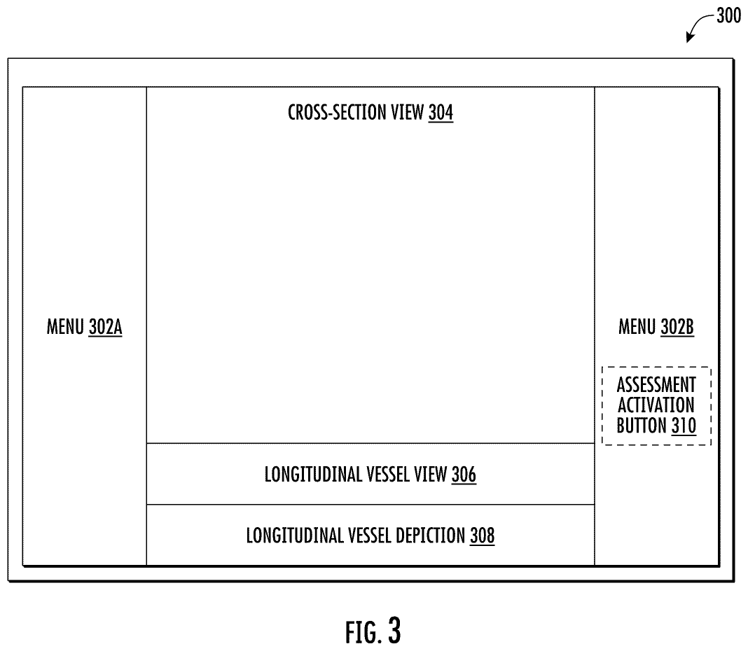

FIG. 2 illustrates an IVUS visualization system 200, according to some embodiments of the present disclosure. In general, IVUS visualization system 200 is a system for processing, annotating, and presenting IVUS images. IVUS visualization system 200 can be implemented in a commercial IVUS system (also referred to as an IVUS guidance system or and IVUS navigation system). For example, IVUS visualization system 200 can be implemented in the AVVIGOR Guidance System available from Boston Scientific®. The present disclosure provides advantages over prior or conventional IVUS systems in that the improved GUI will reduce the time needed for patients to be in treatment. For example, the present disclosure can be implemented in an IVUS system to efficiently communicate, via graphical visualization, ratios of stent expansion along the longitudinal axis of the stent. The provided GUI further allow a user to scrub or pan along the longitudinal axis of the stent and the ratio of stent expansion will be displayed and/or updated dynamically.

With some embodiments, IVUS visualization system 200 could be implemented as part of control system 104. Alternatively, control system 104 could be implemented as part of IVUS visualization system 200. As depicted, IVUS visualization system 200 includes a computing device 202. Optionally, IVUS visualization system 200 includes IVUS imaging system 100 and display 204.

Computing device 202 can be any of a variety of computing devices. In some embodiments, computing device 202 can be incorporated into and/or implemented by a console of display 204. With some embodiments, computing device 202 can be a workstation or server communicatively coupled to IVUS imaging system 100 and/or display 204. With still other embodiments, computing device 202 can be provided by a cloud based computing device, such as, by a computing as a service system accessibly over a network (e.g., the Internet, an intranet, a wide area network, or the like). Computing device 202 can include processor 206, memory 208, input and/or output (I/O) devices 210, network interface 212, and IVUS imaging system acquisition circuitry 214.

The processor 206 may include circuity or processor logic, such as, for example, any of a variety of commercial processors. In some examples, processor 206 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 206 may include graphics processing portions and may include dedicated memory, multiple-threaded processing and/or some other parallel processing capability. In some examples, the processor 206 may be an application specific integrated circuit (ASIC) or a field programmable integrated circuit (FPGA).

The memory 208 may include logic, a portion of which includes arrays of integrated circuits, forming non-volatile memory to persistently store data or a combination of non-volatile memory and volatile memory. It is to be appreciated, that the memory 208 may be based on any of a variety of technologies. In particular, the arrays of integrated circuits included in memory 208 may be arranged to form one or more types of memory, such as, for example, dynamic random access memory (DRAM), NAND memory, NOR memory, or the like.

I/O devices 210 can be any of a variety of devices to receive input and/or provide output. For example, I/O devices 210 can include, a keyboard, a mouse, a joystick, a foot pedal, a display, a touch enabled display, a haptic feedback device, an LED, or the like.

Network interface 212 can include logic and/or features to support a communication interface. For example, network interface 212 may include one or more interfaces that operate according to various communication protocols or standards to communicate over direct or network communication links.

Direct communications may occur via use of communication protocols or standards described in one or more industry standards (including progenies and variants). For example, network interface 212 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like.

Additionally, network interface 212 can include logic and/or features to enable communication over a variety of wired or wireless network standards (e.g., 802.11 communication standards). For example, network interface 212 may be arranged to support wired communication protocols or standards, such as, Ethernet, or the like. As another example, network interface 212 may be arranged to support wireless communication protocols or standards, such as, for example, Wi-Fi, Bluetooth, LTE, 5G, or the like.

The IVUS imaging system acquisition circuitry 214 may include circuity including custom manufactured or specially programmed circuitry configured to receive, send, and/or receive and send signals between IVUS imaging system 100 including electrical signals to and/or from a transducer of an IVUS catheter (e.g., catheter 102, or the like), indications of an IVUS run, indications of a series of IVUS images (e.g., a frame or frames of IVUS images).

Memory 208 can include instructions 216. During operation processor 206 can execute instructions 216 to cause computing device 202 to receive (e.g., from IVUS imaging system 100, or the like) a recording of an “IVUS run” and store the recording as IVUS images 218 in memory 208. For example, processor 206 can execute instructions 216 to receive information elements from IVUS imaging system 100 (e.g., image data structures, electronic signals, etc.) comprising indications of IVUS images captured by catheter 102 while being pulled back, longitudinally, from a distal point to a proximal point of a vessel. It is to be appreciated, that the IVUS images 218 comprise indications of the anatomy and/or structure of vessel, including vessel walls, plaque, and any stents deployed in the vessel.

IVUS images 218 can be stored in a variety of structures or formats that comprise indications. Further, IVUS images 218 will typically include several “frames” or several individual images. Each individual frame represents a snapshot of the IVUS image at a location along the longitudinal axis of the vessel. When represented co-linearly, IVUS images 218 can be used to form a representation of visualization of the vessel.

Processor 206 can further execute instructions 216 to generate assessments 220 based on IVUS images 218. This will be described in greater detail below. However, in general, the assessments can include any of a variety of observations, detections, or inferences about the vessel anatomy and stent deployment and placement. For example, assessments 220 can include, without limitation, vessel boundary detection, lumen boundary detection, plaque burden determination, key frame identification, distances between key frames, stent detection, stent expansion values.

Processor 206 can further execute instructions 216 to generate graphical information elements 222 from IVUS images 218 and assessments 220 and further to generate a GUI 224 comprising the graphical information elements 222 and cause the GUI 224 to be displayed on display 204. Graphical information elements 222 and GUI 224 will be described in greater detail below. However, in general, graphical information elements 222 can comprise visual indications of a stent in a vessel represented by IVUS images 218 and visual indications of a ratio of expansion of the stent (sometimes referred to as ratio of stent expansion or expansion ratio herein) at multiple points along the longitudinal axis of the vessel. Processor 206 can execute instructions 216 to dynamically update the visualization of the expansion ratio.

For example, processor 206 can execute instructions 216 to receive manipulations 226 including inputs received via GUI 224. For example, graphical information elements 222 and GUI 224 can include a visualization of the longitudinal axis of the vessel represented in IVUS images 218 as well as a scrubber (e.g., slider, or the like) indicating a point along the longitudinal axis). Processor 206 can execute instructions 216 to receive indications to move the scrubber and store such indications as manipulations 226. Responsive to receiving manipulations 226, processor 206 can execute instructions 216 to dynamically generate graphical information elements 222 and GUI 224 to visually indicate the stent expansion at the point along the longitudinal axis of the vessel corresponding to the scrubber location.

As used herein, “ratio of stent expansion” means the stent area, at a point along the longitudinal axis of the vessel, over a lumen area. It is to be appreciated that a variety of possible measures of “lumen area” can be used. For example, ideal lumen area, proximal reference (or key frame) lumen area, distal reference lumen area, average of the proximal and distal reference lumen areas, vessel area at the point along the longitudinal axis, proximal reference (or key frame) vessel area, distal reference vessel area, average of the proximal and distal reference vessel areas, or the like. Further, area (e.g., stent area, lumen area, or vessel area) can be derived based on geometric algorithms, vessel, and lumen area models, or inferred via a machine learning model. Specifics of derivation of the stent expansion are beyond the scope of this disclosure and as such, will not be covered in greater detail.

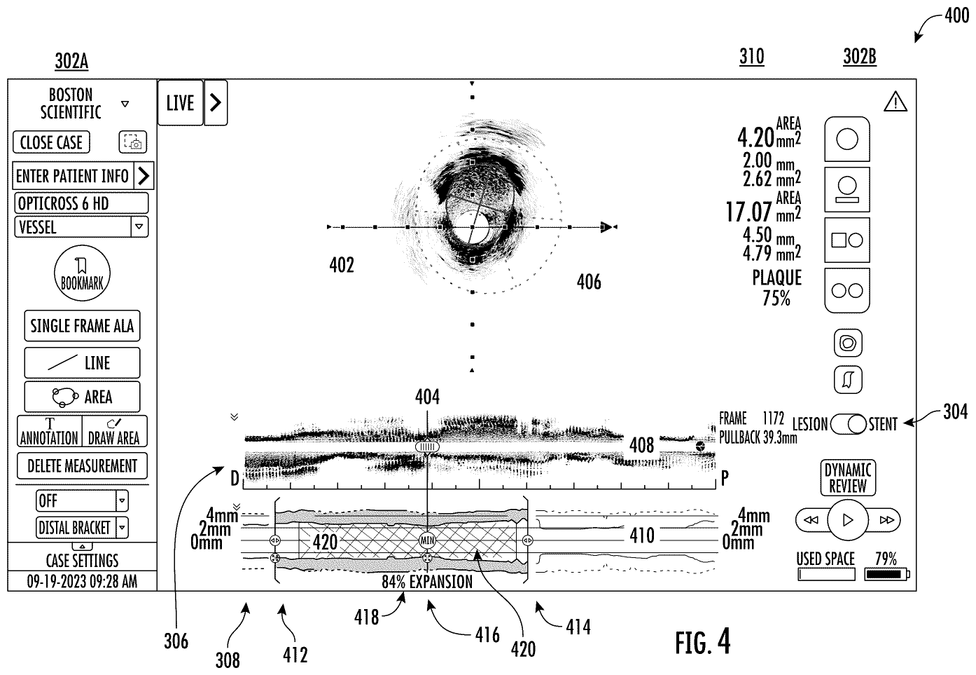

FIG. 3 illustrates a GUI 300, which can be generated according to some embodiments of the present disclosure. For example, GUI 300 can be generated by IVUS imaging system 100 as GUI 224 and displayed on display 204. As depicted, GUI 300 includes several graphical information elements (e.g., graphical information elements 222), such as, menus 302a and 302b, cross-section view 304, and longitudinal vessel depiction 308. With some embodiments, processor 206 can execute instructions 216 to generate GUI 300 once assessment activation button 310. As another example, processor 206 can execute instructions 216 to generate GUI 300 responsive to an automatic stent detection process. For example, with some embodiments, IVUS visualization system 200 can be arranged to automatically detect stents from IVUS images 218 (e.g., via machine learning, image classification, or the like). Responsive to detection of a stent in IVUS images 218, stent expansion ratios can be determined and displayed as outlined herein.

Menus 302a and 302b can comprise several GUI inputs, in various combinations, such as, for example, button(s), drop down menu(s), selection icon(s), toggle(s), slider(s), or the like. Menus 302a and 302b can include GUI input options to select measurement and annotation tools, length tools, modification reset buttons, layout options of the views, annotations, navigation, dynamic review options, status of the computing device, or the like.

Cross-section view 304 can comprise a cross-sectional view of a one (e.g., a single frame, or the like) of IVUS images 218. Said differently, cross-section view 304 can include a two-dimensional (2D) representation of a slice along the axis of the vessel based on one of IVUS images 218 (sometimes referred to as an on-axis or cross-section view). With some embodiments, indications of assessments 220 can be provided via graphical information elements 222 in the cross-section view 304 portion of GUI 300. For example, the vessel and lumen border for the portion of the vessel represented by the frame of IVUS images 218 depicted in cross-section view 304 can be displayed in cross-section view 304.

A detailed description of longitudinal vessel view 306 and longitudinal vessel depiction 308 is provided below. However, in general, longitudinal vessel view 306 includes a view of a slice of the vessel represented by IVUS images 218 along the pullback path, or along the longitudinal axis of the vessel. For example, longitudinal vessel view 306 can include a 2D representations of a slice along the longitudinal axis of the vessel based on the IVUS images 218 (sometimes referred to as a longitudinal view). Similarly, longitudinal vessel depiction 308 includes depictions of the longitudinal vessel view 306, which may in some embodiments be annotated and/or marked up to provide visualizations of a side profile or side view of the characteristics of the vessel and/or stent.

For example, indications of assessments 220 can be provided via graphical information elements 222 in the longitudinal vessel depiction 308 portion of GUI 300. For example, key frames, vessel and lumen borders, and stent expansion ratios at a point along the longitudinal axis of the vessel, and the like can be depicted in longitudinal vessel view 306 and can be displayed in longitudinal vessel depiction 308.

Further, the graphical information elements 222 comprising the longitudinal vessel view 306 and longitudinal vessel depiction 308 portions of GUI 300 can include a navigation slider (e.g., see FIG. 4) to navigate along the longitudinal axis of the vessel. With some embodiments, the image depicted in cross-section view 304 is based on the position of the slider. Further, processor 206 can execute instructions 216 to dynamically update the GUI 300 (e.g., graphical information elements 222, or the like) to indicate the stent expansion ratio based on the position of the slider.

FIG. 4 illustrates a GUI 400, which can be generated according to some embodiments of the present disclosure. For example, GUI 400 can be generated by IVUS visualization system 200 as GUI 224 and displayed on display 204. With some embodiments, responsive to detection of a stent, processor 206 can execute instructions 216 to generate graphical information elements 222 and GUI 400 from graphical information elements 222.

As depicted, GUI 400 includes menu 302a and menu 302b disposed on either sides of (or framing) cross-section view 304, longitudinal vessel view 306, and longitudinal vessel depiction 308. Cross-section view 304 includes depictions or representations of a vessel cross-section 402 (e.g., an on-axis view of the vessel) corresponding to the point in IVUS images 218 at which slider 404 is disposed as well as depictions or representations of borders 406 (e.g., lumen border, vessel border, diameters, etc.).

Longitudinal vessel view 306 includes depictions of a slice of the vessel along the longitudinal axis, as vessel longitudinal slice 408 while longitudinal vessel depiction 308 includes depictions of the vessel longitudinal slice 408 with assessments 220 as vessel profile 410. As noted above, longitudinal vessel depiction 308 can include indications of assessments 220, such as, for example, borders 406, distal bracket end 412, proximal bracket end 414, minimum region 416, stent expansion ratio 418, stent 420, and/or the like. More detailed examples of vessel profile 410 and stent expansion ratio 418 are given below.

Additionally, GUI 300 can include (e.g., as part of menus 302a or 302b) assessment activation button 310, which in this example is a toggle that can be moved to activate and deactivate the visualizations of the stent expansion ratio 418 and stent 420.

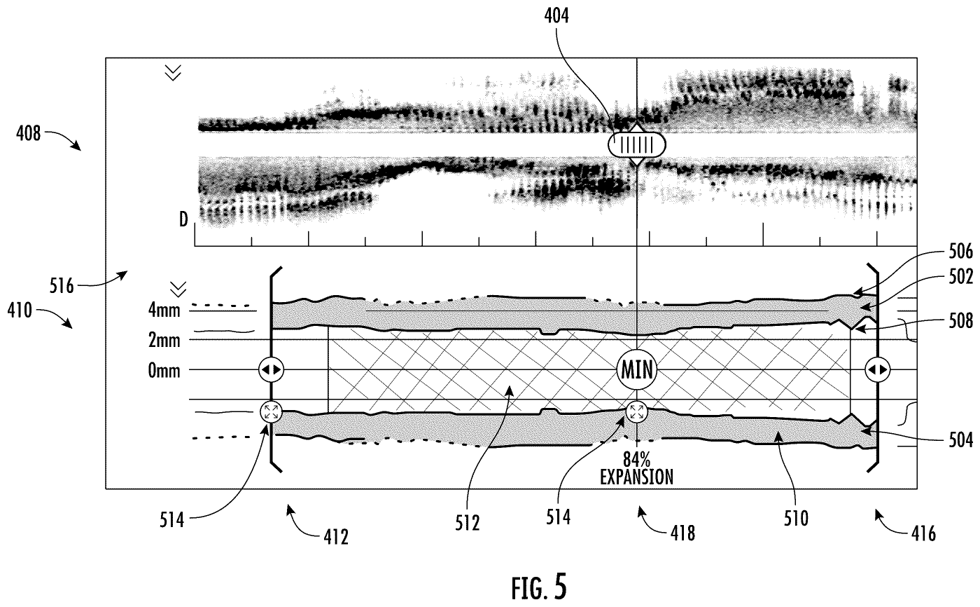

FIG. 5 illustrates a portion of GUI 400 in enlarged detail. For example, this figure illustrates portions of vessel longitudinal slice 408 and vessel profile 410 of GUI 400 as well as a portion of slider 404. As depicted, vessel profile 410 can include longitudinal border profile 502 and longitudinal border profile mirror reflection 504, which each include vessel border 506 and lumen border 508. For example, as outlined above, with some examples, processor 206 can execute instructions 216 to automatically detect the vessel and lumen borders. Further, processor 206 can execute instructions 216 to represent the detected vessel and lumen borders as vessel border 506 and lumen border 508. Further still, processor 206 can execute instructions 216 to mirror a graphical representation of the detected borders to present a more realistic two-dimensional view of the vessel and lumen profile, as depicted in FIG. 4 and FIG. 5.

With some embodiments a GUI 400 can include a scale 516 measured radially from the longitudinal axis 518. As depicted, the longitudinal vessel depiction 308 includes the vessel profile 410, which can include a longitudinal representation of the vessel border 506 and the lumen border 508 (e.g., longitudinal border profile 502) and a mirror of the longitudinal representation (e.g., longitudinal border profile mirror reflection 504) reflected about the longitudinal axis 518.

Additionally, processor 206 can execute instructions 216 to shade, color, or otherwise format the graphical visualization (e.g., line weight, solid line, dashed line, dotted line, area shading, area color, area pattern, etc.) to indicate plaque, confidence of border detection, a detected stent, or the like. For example, the area between vessel border 506 and lumen border 508 on the radial axis and the distal bracket end 412 and minimum region 416 on the longitudinal axis (e.g., shaded area 510) can be shaded a different color than the background of the GUI to indicate a plaque burden. Similarly, portions of the lines indicating the vessel border 506 and lumen border 508 can be solid while other portions can be dashed indicating a confidence in the border detection. Similarly, vessel border 506 and lumen border 508 can be different colors to indicate which is the vessel and which is the lumen. As another example, the stent 420 can be represented as a patterned area (e.g., stent area 512) using a pattern, such as, hatch marks in this example.

With some embodiments, processor 206 can execute instructions 216 to determine the location of the distal bracket ends 412 and 414 based on the distal and proximal ends of the stent 420. For example, processor 206 can execute instructions 216 to place the key frames a specified distance beyond the ends of the stent 420 (e.g., 1 millimeter (mm), 2 mm, between 2 and 6 mm, or the like).

Additionally, processor 206 can be configured to determine an expansion amount (e.g., percentage, ratio, or the like) for the detected stent and display the determined expansion amount in the longitudinal vessel depiction 308. For example, GUI 400 depicts distal bracket ends 412 and 414, which correspond to the proximal and distal key frames, respectively; as well as the stent expansion ratio 418, which corresponds to the expansion ratio at the location of the slider 404. Said differently, the value depicted at stent expansion ratio 418 can be derived (e.g., dynamically, prior to generation of GUI 400, or the like) and the visualization of the expansion amount dynamically updated based on the location or movement of the slider 404.

With some examples, the expansion ratio can be derived based on the minimum stent area (MSA) divided by the lumen area, multiplied by 100, and visualized as a percentage as shown. In some embodiments, the expansion ratio could be visualized as a ratio (e.g., reduced fraction, or the like). In some embodiments, the expansion ratio could be visualized by an indication of whether it exceeds a threshold or not. For example, the expansion ratio could be a first icon or indicator (e.g., green circle, or the like) where the expansion ratio is greater than or equal to a threshold value while in other examples the expansion ration could be a second icon or indicator (e.g., red circle, or the like) where the expansion ratio is less than the threshold value. With some embodiments, the expansion ratio can be visualized as a percentage while the color or the text of the icon depends upon whether the percentage is greater than or less than the threshold as discussed above. In some embodiments, processor 206 can execute instructions 216 to generate graphical information elements 222 and GUI 224 (e.g., GUI 400) to include one or more stent expansion ratio icons 514.

In general, the stent expansion ratio icons 514 can be any of a variety of graphical icons. For example, in the GUIs depicted herein, the stent expansion ratio icon 514 is a circle with four (4) arrows expanding to the outer circumference of the circle to indicate that the expansion ratio is derived based on an area measure (e.g., MSA and lumen area, or the like). Further, the location of the stent expansion ratio icon 514 can be set to indicate the location (e.g., reference frame, or the like) that is used as the lumen area in deriving the expansion ratio (e.g., distal bracket ends 412, proximal bracket end 414, or the like). For example, GUI 400 depicts stent expansion ratio icon 514 on the distal bracket end 412 indicating that the lumen area at the distal bracket end 412 is used to derive the expansion ratio. With some examples, processor 206 can execute instructions 216 to provide a GUI 400 with a drop down menu to select which reference frame (e.g., distal bracket end 412, proximal bracket end 414, etc.) to use in deriving the expansion ratio. Processor 206 can execute instructions 216 to generate GUI 400 with ones of the stent expansion ratio icons 514 on the selected reference frame responsive to a change in the reference frame selection. With some embodiments, processor 206 can execute instructions 216 to default to the distal bracket end 412.

In some examples, stent expansion ratio icons 514 can be disposed on multiple reference frames, such as, to indicate that the area at multiple frames is used to derive the expansion ratio (e.g., average proximal and distal lumen area, or the like). With some embodiments, the stent expansion ratio icons 514 can be colored the same as the lumen border 508 or the vessel border 506 to indicate which border is used in deriving the expansion ratio.

In some examples, one of stent expansion ratio icons 514 can be pinned or fixed to slider 404, such that as the slider 404 is moved the stent expansion ratio icon 514 can follow the location of the slider 404 to indicate that the expansion ratio is derived based on the location of the slider 404 (e.g., stent area at the location, or the like). In further examples, the stent expansion ratio icon 514 pinned to the slider 404 can follow the profile (e.g., lumen border 508, vessel border 506, etc.) as the slider 404 is moved along the longitudinal axis of the longitudinal vessel depiction 308.

With some examples, processor 206 can execute instructions 216 to provide a GUI 400 with the location of the slider 404 defaulting to the minimum region 416.

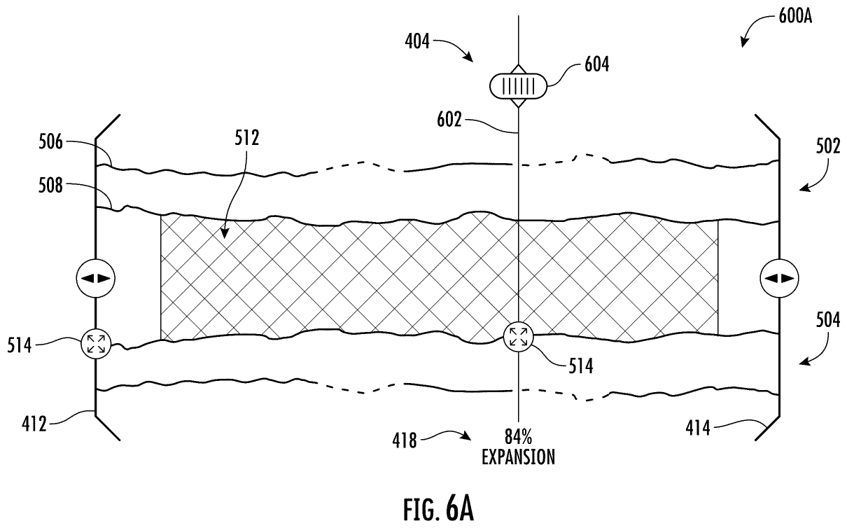

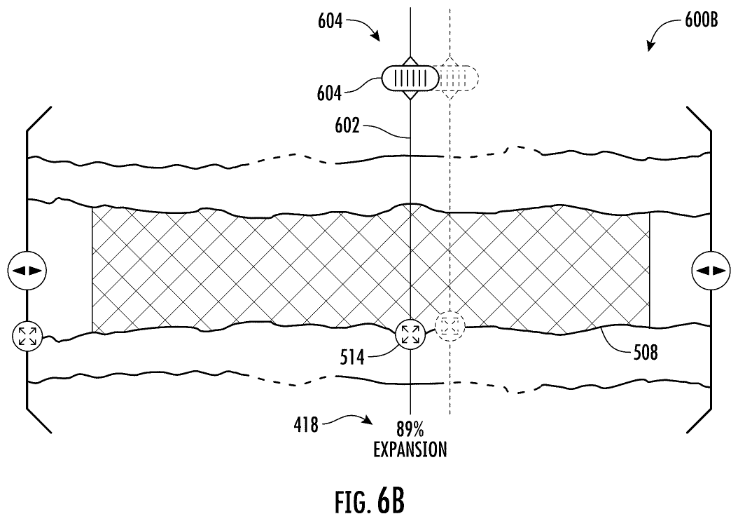

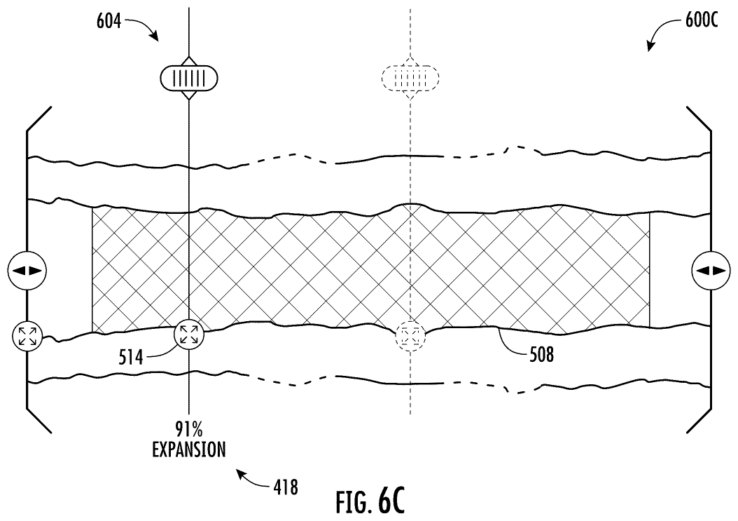

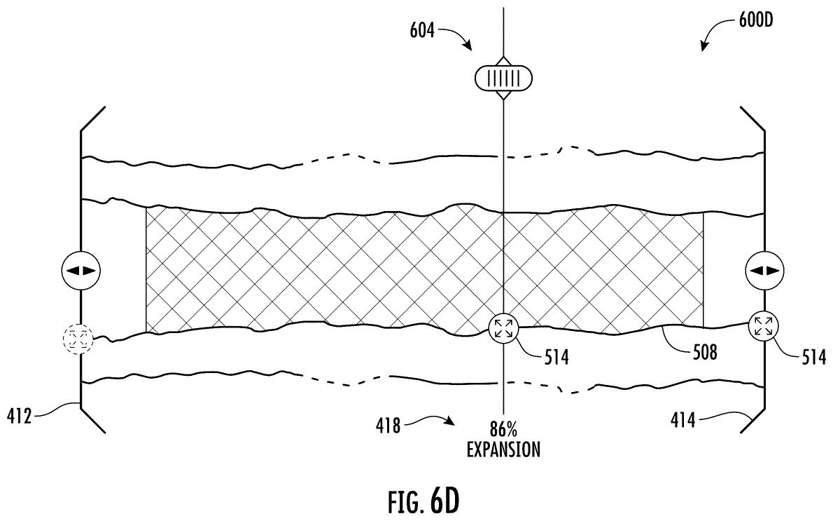

FIG. 6A to FIG. 6D illustrate a longitudinal border profile (e.g., longitudinal vessel depiction 308, or the like) that can be part of a GUI as described herein. For example, the depictions of these figures can be part of GUI 224, which can be dynamically updated based on selections or interactions with the GUI as described herein. For example, FIG. 6A to FIG. 6D illustrate a longitudinal vessel depiction where the slider 404 is moved, resulting in a dynamic update to the stent expansion ratio icon 514 and the stent expansion ratio 418. Further, these figures depict an example where the reference frame for deriving the expansion ratio is moved from the distal bracket end 412 to the proximal bracket end 414, resulting in a dynamic change in the stent expansion ratio 418.

Turning more particularly to FIG. 6A, longitudinal vessel depiction 600a is shown. As depicted, longitudinal vessel depiction 600a includes several graphical information elements 222 or GUI components, such as, for example, distal bracket end 412 and proximal bracket end 414 within which longitudinal border profile 502 and longitudinal border profile mirror reflection 504 are disposed. Longitudinal border profile 502 and longitudinal border profile mirror reflection 504 each include vessel border 506 and lumen border 508. Longitudinal vessel depiction 600a further includes slider 404 located at a point along the longitudinal axis of the vessel. Stent expansion ratio icons 514 are also depicted. In this example, one of the stent expansion ratio icons 514 is disposed on the distal bracket end 412 (e.g., indicating that the distal key frame is used to derive the expansion ratio) while the other stent expansion ratio icon 514 is pinned to the slider 404. In some embodiments, graphical information elements 222 for slider 404 can comprise a line marking the location along the longitudinal axis of the vessel, such as, location marker 602 and a slider button 604 that a user can interact with to change the location (e.g., scrub over the vessel, or the like).

In some examples as depicted in this figure, the stent expansion ratio icon 514 pinned to the slider 404 can be also pinned to the lumen border 508.

Turning to FIG. 6B, longitudinal vessel depiction 600b is depicted, which shows the longitudinal vessel depiction 600a with the difference being that the slider 404 has been moved and the longitudinal vessel depiction 600a dynamically updated to show movement of the slider 404, stent expansion ratio icon 514 and a change in the expansion ratio. For example, longitudinal vessel depiction 600b shows the stent expansion ratio icon 514 pinned to the slider 404 moved along with the location marker 602 and slider button 604 to a new location. Further, as illustrated, the stent expansion ratio icon 514 pinned to the slider 404 has tracked the lumen border 508. That is, processor 206 can execute instructions 216 to dynamically generate GUI 224 where the stent expansion ratio icon 514 pinned to the slider 404 moves in x and y directions to track the both the location of slider 404 (e.g., location marker 602) and the lumen border 508. Further, the stent expansion ratio 418 can be updated to reflect the value of the expansion ratio at the new location of the slider 404.

It is noted that the shadow (e.g., dashed lines) of slider 404 and stent expansion ratio icon 514 are depicted in longitudinal vessel depiction 600b for purposes of illustration of movement of slider 404 with respect to longitudinal vessel depiction 600a. In practice, GUI 224 may only include one visualization of the slider 404.

Turning to FIG. 6C, longitudinal vessel depiction 600c is depicted, which shows the longitudinal vessel depiction 600b with the difference being that the slider 404 has been further moved and the longitudinal vessel depictions 600a and/or 600b dynamically updated to show movement of the slider 404, stent expansion ratio icon 514 and a change in the expansion ratio. Like longitudinal vessel depiction 600b, longitudinal vessel depiction 600c shows the stent expansion ratio icon 514 pinned to the slider 404 moved along with the location marker 602 and slider button 604 to a new location. Further, as illustrated, the stent expansion ratio icon 514 pinned to the slider 404 has tracked the lumen border 508. Further, the value depicted (or visualized) via stent expansion ratio 418 can be updated to reflect the expansion ratio at the new location of the slider 404.

It is noted that the shadow (e.g., dashed lines) of slider 404 and stent expansion ratio icon 514 are depicted in longitudinal vessel depictions 600b and 600c for purposes of illustration of movement of slider 404 with respect to longitudinal vessel depictions 600a and/or 600b. In practice, however, GUI 224 may only include one visualization of the slider 404 and will not include the shadow visualization.

Turning to FIG. 6D, longitudinal vessel depiction 600d is depicted, which shows the longitudinal vessel depiction 600a with the difference being that the stent expansion ratio icon 514 disposed on the distal bracket end 412 has moved to the proximal bracket end 414 indicating that the key frame at the location of proximal bracket end 414 is used to derive the expansion ratio as outlined herein. Processor 206 can execute instructions 216 to dynamically generate GUI 224 where the stent expansion ratio icon 514 can be moved from distal bracket end 412 to proximal bracket end 414 (or vice versa) responsive to input received via the GUI 224 (or another input device, such as I/O devices 210). For example, as detailed above, a user can select, via GUI 224, which key frame to use to derive the expansion ratio. Processor 206 can execute instructions 216 to dynamically update GUI 224 to visualize this selection, for example, by moving stent expansion ratio icon 514 from distal bracket end 412 to proximal bracket end 414 as shown in longitudinal vessel depiction 600d. Processor 206 can execute instructions 216 to derive the expansion ratio based on the selected reference frame (e.g., proximal bracket end 414 as shown in FIG. 6D) and further to update the value depicted in stent expansion ratio 418 to correspond to the newly derived expansion ratio.

Again, a shadow (e.g., dashed lines) of stent expansion ratio icon 514 is depicted in longitudinal vessel depiction 600d for purposes of illustration of movement of the stent expansion ratio icon 514 to indicate the selection of which key frame to use in deriving stent expansion ratio 418.

With some embodiments, processor 206 can execute instructions 216 to dynamically update GUI 224 responsive to received input to move the locations of distal bracket end 412 and/or proximal bracket end 414. That is, distal bracket end 412 and proximal bracket end 414 can be movable via user input (e.g., via I/O devices 210) and the GUI 224 dynamically updated to reflect the change in location of the distal bracket end 412 and/or proximal bracket end 414. As discussed herein, a change in the location of distal bracket end 412 and/or proximal bracket end 414 may result in a change in the stent expansion. As such, the value depicted via stent expansion ratio 418 can be dynamically updated.

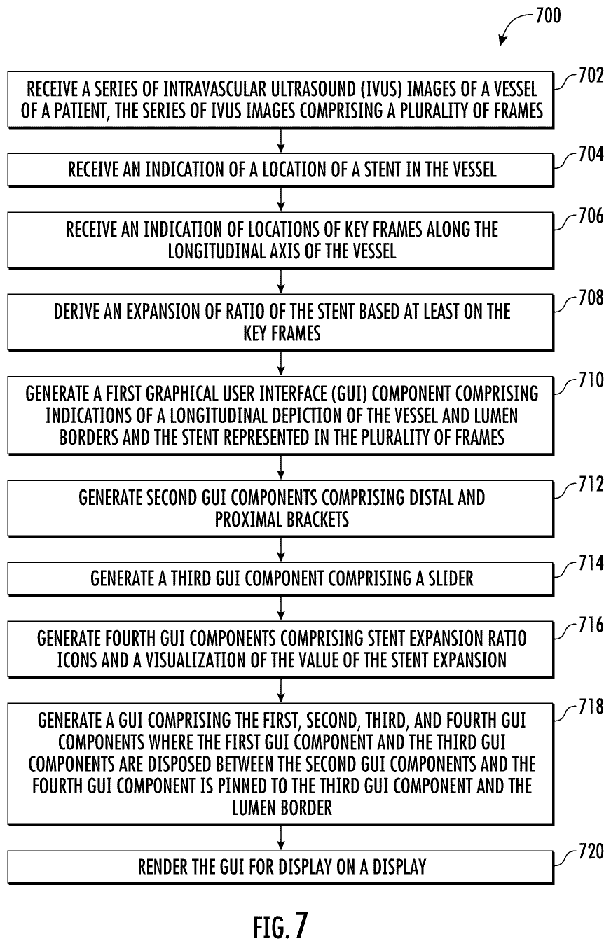

FIG. 7 illustrates a logic flow 700 to generate a GUI, according to some embodiments of the present disclosure. The logic flow 700 can be implemented by IVUS visualization system 200 and will be described with reference to IVUS visualization system 200 for clarity of presentation. However, it is noted that logic flow 700 could also be implemented by an IVUS system different than IVUS visualization system 200.

Logic flow 700 can begin at block 702. At block 702 “receive a series of intravascular ultrasound (IVUS) images of a vessel of a patient, the series of IVUS images comprising a plurality of frames” a series of IVUS images captured via an IVUS catheter percutaneously inserted in a vessel of a patent can be received. For example, information elements comprising indications of IVUS images 218 can be received from IVUS imaging system 100 where catheter 102 is (or was) percutaneously inserted into a vessel. The IVUS images 218 can comprise frames of images representative of images captured while the catheter 102 is pulled back from a distal location in the vessel to a proximal location in the vessel. Processor 206 can execute instructions 216 to receive information elements comprising indications of IVUS images 218 from IVUS imaging system 100, or directly from catheter 102 as may be the case.

Continuing to block 704 “receive an indication of a location of a stent in the vessel” an indication of a location of a stent in the vessel captured in the IVUS images can be received. In some examples, an indication of the frames where the stent is identified are received. For example, processor 206 can execute instructions 216 to receive an indication of the frames in IVUS images 218 where the stent 420 is identified or present. In some examples, processor 206 can execute instructions 216 to detect the stent based on a machine learning (ML) model (e.g., an image classification model, or the like). For example, such an ML model could be trained to infer frames of a series of IVUS image frames where a stent is found (e.g., using supervised learning, unsupervised learning, or the like). The trained ML model could be deployed and used to infer stent 420 locations in IVUS images 218. For example, processor 206 could execute instructions 216 to infer stent 420 from IVUS images 218 using an ML model (not shown). Processor 206 can execute instructions 216 to store the stent location (e.g., frames where the stent was detected, or the like) as assessments 220 in memory 208.

Continuing to block 706 “receive an indication of locations of key frames along the longitudinal axis of the vessel” an indication of a locations of key frames (e.g., distal, and proximal key frames, minimum key frame, etc.) along the longitudinal axis of the vessel can be received. It is noted that a variety of algorithms, ML models, and key frame detection schemes exist to identify key frames. The specific mechanism to identify the location of the key frames is beyond the scope of this disclosure. However, derivation of the expansion ratio is based in part on the key frame, and particularly the area at the key frame locations. As such, location of the key frames is necessary to implementing the disclosure. For example, processor 206 can execute instructions 216 to identify key frames from IVUS images 218 and to store the locations (e.g., frame number, or the like) of the key frames in memory 208 and store the key frames and/or locations as assessments 220 in memory 208. As a specific example, the initial expansion ratio can be derived as the stent area at the minimum key frame divided by the lumen area at the distal key frame, where the ratio can be multiplied by 100 to represent a percentage value.

Continuing to block 708 “derive an expansion ratio of the stent based at least on the key frames” an expansion ratio of the stent can be derived using, in part, the key frames identified by locations received at block 706. For example, processor 206 can execute instructions 216 to determine lumen and stent areas at key frame locations and to derive the expansion ratio accordingly. With some embodiments, processor 206 can execute instructions 216 to implement an automated (e.g., algorithmic, ML based, or the like) border detection process to identify the vessel and lumen borders and to derive the expansion ratio accordingly. Processor 206 can execute instructions 216 to store the expansion ration (as well as lumen and vessel borders, or the like) as assessments 220 in memory 208.

Continuing to block 710 “generate a first graphical user interface (GUI) component comprising indications of a longitudinal depiction of the vessel and lumen borders and the stent represented in the plurality of frames” a first GUI component comprising indications of a longitudinal depiction of the vessel and the stent represented in the IVUS images is generated. For example, processor 206 can execute instructions 216 to generate longitudinal border profile 502 and longitudinal border profile mirror reflection 504 including visualizations of vessel border 506 and lumen border 508 as well as stent area 512. Processor 206 can execute instructions 216 to store the first GUI component as graphical information elements 222 in memory 208.

Continuing to block 712 “generate second GUI components comprising distal and proximal brackets” second GUI components comprising indications of distal and proximal brackets are generated. For example, processor 206 can execute instructions 216 to generate distal bracket end 412 and proximal bracket end 414. Processor 206 can execute instructions 216 to store the second GUI components as graphical information elements 222 in memory 208. Continuing to block 714 “generate a third GUI component comprising a slider” third GUI components comprising a slider is generated. For example, processor 206 can execute instructions 216 to generate slider 404. Processor 206 can execute instructions 216 to store the third GUI component as graphical information elements 222 in memory 208. Continuing to block 716 “generate fourth GUI components comprising stent expansion ratio icons and a visualization of the value of the stent expansion” fourth GUI components comprising a visualization of the value of the expansion ratio and stent expansion ratio icons are generated. For example, processor 206 can execute instructions 216 to generate stent expansion ratio 418 and stent expansion ratio icon 514. Processor 206 can execute instructions 216 to store the fourth GUI components as graphical information elements 222 in memory 208.

Continuing to block 718 “generate a GUI comprising the first, second, third, and fourth GUI components where the first GUI component and the third GUI components are disposed between the second GUI components and the fourth GUI component is pinned to the third GUI component and the lumen border” a GUI comprising the first, second, third, and fourth GUI components where the first and third GUI components are disposed between the second GUI components and where the fourth GUI component is pinned to the third GUI component and the lumen border of the first GUI component. For example, processor 206 can execute instructions 216 to generate GUI 224. GUI 224 can include longitudinal border profile 502, longitudinal border profile mirror reflection 504 and stent area 512 with vessel border 506 and lumen border 508 visualized in the profile 502 and reflection 504 (e.g., first GUI component). GUI can further include slider 404 (e.g., third GUI component). In GUI 224, profile 502, reflection 504, stent area 512, and slider 404 can be disposed between distal bracket end 412 and proximal bracket end 414 (e.g., second GUI components). For example, the distal bracket end 412 and proximal bracket end 414 can bookend the longitudinal border profile 502 and longitudinal border profile mirror reflection 504 while the slider 404 is disposed at a location of the minimum key frame. Further, in GUI 224, the stent expansion ratio 418 and the stent expansion ratio icon 514 (e.g., fourth GUI component) can be pinned to the slider 404 (e.g., third GUI component). Stent expansion ratio 418 can be pinned to slider 404 (e.g., pinned to location marker 602 of slider 404) while stent expansion ratio icon 514 (or one of stent expansion ratio icons 514) can be pinned to the slider 404 (e.g., location marker 602 of slider 404) and the lumen border 508 at the location of marker 602.

Continuing to block 720 “render the GUI for display on a display” the GUI can be rendered for display. For example, processor 206 can execute instructions 216 to render the graphical information elements 222 (e.g., GUI components) and GUI 224 for display on display 204. Further, processor 206 can execute instructions 216 to send instructions (e.g., data structures, image frames, etc.) to display 204 to cause display 204 to display the GUI 224.

In some embodiments, logic flow 700 can further include blocks (not shown) to generate other GUI components, such as, for example, to generate GUI component comprising indications of a cross-section view of a one of the IVUS frames; GUI components comprising indications of a menus including various menu options (e.g., expansion ratio reference frame selection, expansion ratio activation button, etc.); GUI components comprising a longitudinal view of the vessel; and/or GUI components comprising visualizations of assessments (e.g., lumen area, vessel area, borders, border diameters, etc.). Processor 206 can execute instructions 216 to generate GUI 224 as outlined above with respect to blocks block 710 to block 718 and can additionally include other GUI components (e.g., menu 302a, menu 302b, cross-section view 304, longitudinal vessel view 306, assessment activation button 310, vessel cross-section 402, borders 406, vessel longitudinal slice 408, minimum region 416, and/or the like).

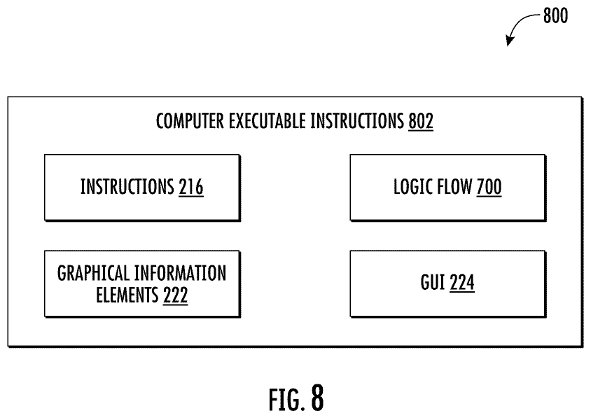

FIG. 8 illustrates computer-readable storage medium 800. Computer-readable storage medium 800 may comprise any non-transitory computer-readable storage medium or machine-readable storage medium, such as an optical, magnetic or semiconductor storage medium. In various embodiments, computer-readable storage medium 800 may comprise an article of manufacture. In some embodiments, computer-readable storage medium 800 may store computer executable instructions 802 with which circuitry (e.g., processor 106, processor 206, IVUS imaging system acquisition circuitry 214, and the like) can execute. For example, computer executable instructions 802 can include instructions to implement operations described with respect to instructions 216, logic flow 700, graphical information elements 222, and/or GUI 224. Examples of computer-readable storage medium 800 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 802 may include any suitable type of code, such as source code, compiled code, interpreted code, executable code, static code, dynamic code, object-oriented code, visual code, and the like.

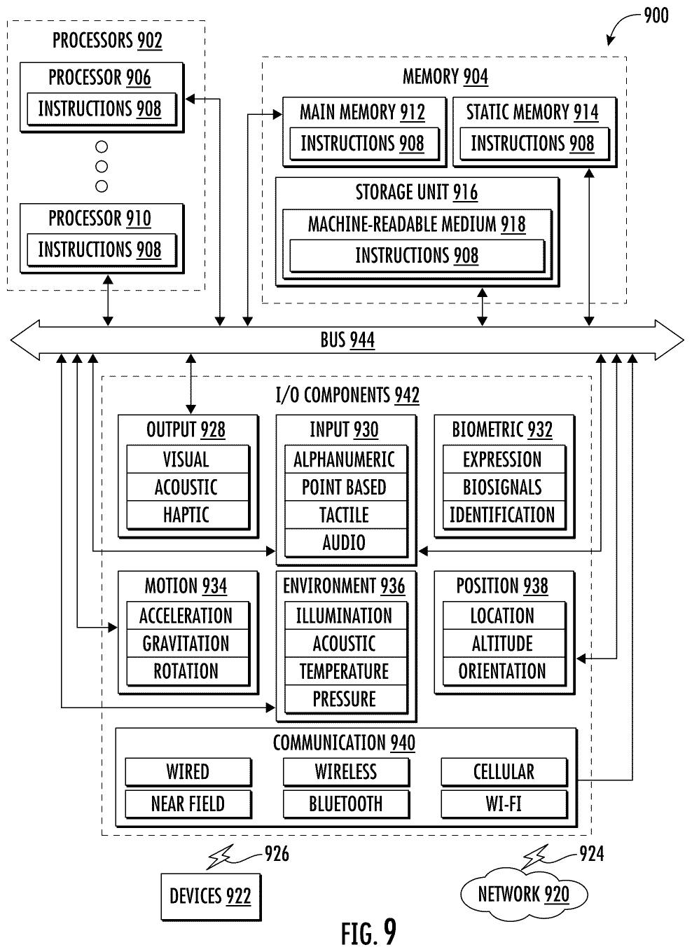

FIG. 9 illustrates a diagrammatic representation of a machine 900 in the form of a computer system within which a set of instructions may be executed for causing the machine to perform any one or more of the methodologies discussed herein. More specifically, FIG. 9 shows a diagrammatic representation of the machine 900 in the example form of a computer system, within which instructions 908 (e.g., software, a program, an application, an applet, an app, or other executable code) for causing the machine 900 to perform any one or more of the methodologies discussed herein may be executed. For example, the instructions 908 may cause the machine 900 to execute logic flow 700 of FIG. 7, instructions 216 of FIG. 2. More generally, the instructions 908 may cause the machine 900 to generate GUIs with functionality and behavior as described herein during a PCI procedure (e.g., peri-PCI or post-PCI) using IVUS. It is noted that the present disclosure provides specific and discrete implementations of GUI representations and behavior that is a significant improvement over the prior art. In particular, the present disclosure provides an improvement to computing technology in that GUIs provide greater visibility of expansion ration along the longitudinal axis of the vessel to provide a physician with options to identify potential needed procedures (e.g., balloon dilation of the stent) to provide and/or ensure higher clinical outcomes from PCI procedures that include stent placement. For example, stent procedures where the stent expansion is greater than or equal to 80% have been found to be associated with better clinical outcomes. Accordingly, the present disclosure provides physicians a more efficient tool to visualize stent expansion. This can lead to shorter PCI procedures and/or reduction in post-PCI procedures, which itself can lead to higher clinical outcomes and better patient safety.

The instructions 908 transform the general, non-programmed machine 900 into a particular machine 900 programmed to carry out the described and illustrated functions in a specific manner. In alternative embodiments, the machine 900 operates as a standalone device or may be coupled (e.g., networked) to other machines. In a networked deployment, the machine 900 may operate in the capacity of a server machine or a client machine in a server-client network environment, or as a peer machine in a peer-to-peer (or distributed) network environment. The machine 900 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 908, sequentially or otherwise, that specify actions to be taken by the machine 900. Further, while only a single machine 900 is illustrated, the term “machine” shall also be taken to include a collection of machines 900 that individually or jointly execute the instructions 908 to perform any one or more of the methodologies discussed herein.

The machine 900 may include processors 902, memory 904, and I/O components 942, which may be configured to communicate with each other such as via a bus 944. In an example embodiment, the processors 902 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 906 and a processor 910 that may execute the instructions 908. The term “processor” is intended to include multi-core processors that may comprise two or more independent processors (sometimes referred to as “cores”) that may execute instructions contemporaneously. Although FIG. 9 shows multiple processors 902, the machine 900 may include a single processor with a single core, a single processor with multiple cores (e.g., a multi-core processor), multiple processors with a single core, multiple processors with multiples cores, or any combination thereof.

The memory 904 may include a main memory 912, a static memory 914, and a storage unit 916, both accessible to the processors 902 such as via the bus 944. The main memory 904, the static memory 914, and storage unit 916 store the instructions 908 embodying any one or more of the methodologies or functions described herein. The instructions 908 may also reside, completely or partially, within the main memory 912, within the static memory 914, within machine-readable medium 918 within the storage unit 916, within at least one of the processors 902 (e.g., within the processor's cache memory), or any suitable combination thereof, during execution thereof by the machine 900.

The I/O components 942 may include a wide variety of components to receive input, provide output, produce output, transmit information, exchange information, capture measurements, and so on. The specific I/O components 942 that are included in a particular machine will depend on the type of machine. For example, portable machines such as mobile phones will likely include a touch input device or other such input mechanisms, while a headless server machine will likely not include such a touch input device. It will be appreciated that the I/O components 942 may include many other components that are not shown in FIG. 9. The I/O components 942 are grouped according to functionality merely for simplifying the following discussion and the grouping is in no way limiting. In various example embodiments, the I/O components 942 may include output components 928 and input components 930. The output components 928 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 930 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 942 may include biometric components 932, motion components 934, environmental components 936, or position components 938, among a wide array of other components. For example, the biometric components 932 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 934 may include acceleration sensor components (e.g., accelerometer), gravitation sensor components, rotation sensor components (e.g., gyroscope), and so forth. The environmental components 936 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 938 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 942 may include communication components 940 operable to couple the machine 900 to a network 920 or devices 922 via a coupling 924 and a coupling 926, respectively. For example, the communication components 940 may include a network interface component or another suitable device to interface with the network 920. In further examples, the communication components 940 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 922 may be another machine or any of a wide variety of peripheral devices (e.g., a peripheral device coupled via a USB).

Moreover, the communication components 940 may detect identifiers or include components operable to detect identifiers. For example, the communication components 940 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 940, such as location via Internet Protocol (IP) geolocation, location via Wi-Fi® signal triangulation, location via detecting an NFC beacon signal that may indicate a particular location, and so forth.

The various memories (i.e., memory 904, main memory 912, static memory 914, and/or memory of the processors 902) and/or storage unit 916 may store one or more sets of instructions and data structures (e.g., software) embodying or utilized by any one or more of the methodologies or functions described herein. These instructions (e.g., the instructions 908), when executed by processors 902, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 920 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 920 or a portion of the network 920 may include a wireless or cellular network, and the coupling 924 may be a Code Division Multiple Access (CDMA) connection, a Global System for Mobile communications (GSM) connection, or another type of cellular or wireless coupling. In this example, the coupling 924 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 908 may be transmitted or received over the network 920 using a transmission medium via a network interface device (e.g., a network interface component included in the communication components 940) and utilizing any one of several well-known transfer protocols (e.g., hypertext transfer protocol (HTTP)). Similarly, the instructions 908 may be transmitted or received using a transmission medium via the coupling 926 (e.g., a peer-to-peer coupling) to the devices 922. The terms “transmission medium” and “signal medium” mean the same thing and may be used interchangeably in this disclosure. The terms “transmission medium” and “signal medium” shall be taken to include any intangible medium that can store, encoding, or carrying the instructions 908 for execution by the machine 900, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An intravascular ultrasound (IVUS) system, comprising:
a processor; and
a memory storage device coupled to the processor, the memory storage device comprising instructions executable by the processor, which when executed cause the processor to:
receive a series of IVUS images of a vessel of a patient, the series of IVUS images comprising a plurality of frames,
receive an indication of a location of a stent in the vessel,
receive an indication of a lumen border,
receive designation of at least two of the plurality of frames as key frames,
derive an expansion ratio of the stent based at least on the key frames,
generate a graphical user interface (GUI) comprising:
a longitudinal vessel depiction comprising at least a portion of the lumen border and the stent,
a distal bracket and a proximal bracket,
a slider,
a stent expansion ratio icon, and
a visualization of the expansion ratio, and
render the GUI for display on a display,
wherein the longitudinal vessel depiction and the slider are disposed between the distal bracket and the proximal bracket.

2. The IVUS system of claim 1, wherein the slider comprises a location marker and a slider button, wherein the location marker is a line substantially perpendicular to a longitudinal axis of the vessel, and wherein the stent expansion ratio icon is disposed on the location marker at a point where the location marker intersects the lumen border.

3. The IVUS system of claim 2, wherein the longitudinal vessel depiction comprises a longitudinal representation of a vessel border and the lumen border and a mirror of the longitudinal representation of the vessel border and the lumen border reflected about the longitudinal axis of the vessel.

4. The IVUS system of claim 3, wherein the key frames comprise an end key frame and minimum area key frame and wherein the instructions when executed by the processor to derive the expansion ratio of the stent cause the processor to:
derive an area of the lumen at the end key frame;
derive an area of the stent at the minimum area key frame; and
compute a quotient of the area of the stent divided by the area of the lumen.

5. The IVUS system of claim 4, wherein the GUI comprises a visualization of a value of the expansion ratio.

6. The IVUS system of claim 5, wherein the slider is disposed at a first point on the longitudinal axis of the vessel, wherein the first point corresponds to the minimum area key frame, and wherein the instructions when executed by the processor further cause the processor to:
receive, from a user of the IVUS system, an indication to move the slider from the point to a second point on the longitudinal axis of the vessel, the second point different from the first point;
derive an updated expansion ratio of the stent based on an area of the stent at the second point and the area of the lumen; and
generate an updated GUI, wherein in the updated GUI the location marker of the slider intersects the lumen border at the second point on the longitudinal axis of the vessel, wherein the stent expansion ratio icon is disposed at the intersection of the location marker and the lumen border, and wherein the visualization of the value of the expansion ratio reflects the updated expansion ratio.

7. The IVUS system of claim 5, wherein the end key frame is a distal key frame, wherein the stent expansion ratio icon is a first stent expansion ratio icon, and wherein the GUI comprises a second stent expansion ratio icon that is disposed at an intersection of the lumen border and the distal bracket.

8. The IVUS system of claim 7, wherein the key frames comprise a proximal key frame and wherein the instructions when executed by the processor further cause the processor to:
receive, from a user of the IVUS system, an indication to utilize the proximal key frame in deriving the expansion ratio;
derive an updated expansion ratio of the stent based on the area of the stent at the minimum key frame and an area of the lumen at the proximal key frame; and
generate an updated GUI, wherein in the updated GUI the visualization of the value of the expansion ratio reflects the updated expansion ratio.

9. The IVUS system of claim 8, wherein the visualization of the value of the expansion ratio depicts a percent.

10. At least one machine readable storage device, comprising a plurality of instructions that in response to being executed by a processor of an intravascular ultrasound (IVUS) system cause the processor to:
receive a series of IVUS images of a vessel of a patient, the series of IVUS images comprising a plurality of frames;
receive an indication of a location of a stent in the vessel;
receive an indication of a lumen border;
receive designation of at least two of the plurality of frames as key frames;
derive an expansion ratio of the stent based at least on the key frames;
generate a graphical user interface (GUI) comprising:
a longitudinal vessel depiction comprising at least a portion of the lumen border and the stent,
a distal bracket and a proximal bracket,
a slider,
a stent expansion ratio icon, and
a visualization of the expansion ratio; and
render the GUI for display on a display,
wherein the longitudinal vessel depiction and the slider are disposed between the distal bracket and the proximal bracket.

11. The at least one machine readable storage device of claim 10, wherein the slider comprises a location marker and a slider button, wherein the location marker is a line substantially perpendicular to a longitudinal axis of the vessel, and wherein the stent expansion ratio icon is disposed on the location marker at a point where the location marker intersects the lumen border.

12. The at least one machine readable storage device of claim 11, wherein the longitudinal vessel depiction comprises a longitudinal representation of a vessel border and the lumen border and a mirror of the longitudinal representation of the vessel border and the lumen border reflected about the longitudinal axis of the vessel.

13. The at least one machine readable storage device of claim 12, wherein the GUI depicts the lumen border in a first color, the vessel border in a second color different from the first color, and the stent expansion ratio icon in the second color.

14. The at least one machine readable storage device of claim 10, wherein the instructions when executed by the processor to receive the indication of the location of the stent in the vessel cause the processor to infer for each frame of the plurality of frames, whether a stent is represented in the frame using a machine learning (ML model).

15. The at least one machine readable storage device of claim 14, wherein the instructions when executed by the processor to receive the indication of the lumen border cause the processor to identify for each frame of the plurality of frames, a diameter of the lumen border at the frame.

16. The at least one machine readable storage device of claim 15, wherein the instructions when executed by the processor to receive designations of at least two of the plurality of frames as key frames cause the processor to:
identify as a distal key frame a one of the plurality of frames distal to a most distal one of the plurality of frames in which the stent is represented;
identify as a proximal key frame a one of the plurality of frames proximal to a most proximal one of the plurality of frames in which the stent is represented;
derive a lumen area for each frame of the plurality of frames between the distal key frame and the proximal key frame; and
identify as a minimum area key frame a one of the plurality of frames between the distal key frame and the proximal key frame with the smallest lumen area.

17. A method, comprising:
receiving, at a processor for an intravascular ultrasound (IVUS) system, a series of IVUS images of a vessel of a patient, the series of IVUS images comprising a plurality of frames;
receiving, at the processor, an indication of a location of a stent in the vessel;
receiving, at the processor, an indication of a lumen border;
receiving, at the processor, designation of at least two of the plurality of frames as key frames;
deriving, at the processor, an expansion ratio of the stent based at least on the key frames;
generating, at the processor, a graphical user interface (GUI) comprising:
a longitudinal vessel depiction comprising at least a portion of the lumen border and the stent,
a distal bracket and a proximal bracket,
a slider,
a stent expansion ratio icon, and
a visualization of the expansion ratio; and
rendering the GUI for display on a display,
wherein the longitudinal vessel depiction and the slider are disposed between the distal bracket and the proximal bracket.

18. The method of claim 17, wherein the slider comprises a location marker and a slider button, wherein the location marker is a line substantially perpendicular to a longitudinal axis of the vessel, and wherein the stent expansion ratio icon is disposed on the location marker at a point where the location marker intersects the lumen border.

19. The method of claim 18, wherein the longitudinal vessel depiction comprises a longitudinal representation of a vessel border and the lumen border and a mirror of the longitudinal representation of the vessel border and the lumen border reflected about the longitudinal axis of the vessel.

20. The method of claim 19, wherein the key frames comprise an end key frame and minimum area key frame and wherein deriving the expansion ratio of the stent comprises:
deriving an area of the lumen at the end key frame;
deriving an area of the stent at the minimum area key frame; and
computing a quotient of the area of the stent divided by the area of the lumen, and wherein the GUI comprises a visualization of a value of the expansion ratio.
